(19) United States
(12) Patent Application Publication
(10) Pub. No.: US 2026/0146038 A1
(43) Pub. Date: May 28, 2026

(54) NOVEL SALT OF DIMETHYL-2,3-DIHYDRO-1H-INDENE DERIVATIVE AND PROCESSES FOR PREPARING THE SAME

(71) Applicants: YUHAN CORPORATION, Seoul (KR); GREEN CROSS CORPORATION, Yongin-si, Gyeonggi-do (KR)

(72) Inventors: Jeong-Ki KANG, Suwon-si, Gyeonggi-do (KR); Hyun JU, Hwaseong-si, Gyeonggi-do (KR); Kyung-Min KO, Yongin-si, Gyeonggi-do (KR); Jung-Ae LEE, Seoul (KR); Yoon-Ah HWANG, Suwon-si, Gyeonggi-do (KR); Se-Yong KIM, Yongin-si, Gyeonggi-do (KR); Jun-Sup LEE, Yongin-si, Gyeonggi-do (KR); Seung-Yub SHIN, Seongnam-si, Gyeonggi-do (KR)

(73) Assignees: YUHAN CORPORATION, Seoul (KR); GREEN CROSS CORPORATION, Yongin-si, Gyeonggi-do (KR)

(21) Appl. No.: 19/123,246

(22) PCT Filed: Oct. 23, 2023
(86) PCT No.: PCT/KR2023/016439

§ 371 Date: Apr. 22, 2025

(30) Foreign Application Priority Data
Oct. 24, 2022 (KR) 10-2022-0137675

(51) Int. Cl. C07D 453/02

(52) U.S. Cl. CPC C07D 453/02; C07B 2200/13

(57) ABSTRACT

The present invention provides a novel salt of (S)-quinuclidin-3-yl ((R)-5-(3-chloro-4-isopropoxyphenyl)-2,2-di-methyl-2,3-dihydro-1H-inden-1-yl)carbamate, i.e., (1S)-(+)-10-camphorsulfonic acid salt thereof and a process for preparing the same. The (1S)-(+)-10-camphorsulfonic acid salt of (S)-quinuclidin-3-yl ((R)-5-(3-chloro-4-isopropoxyphenyl)-2,2-dimethyl-2,3-di-hydro-1H-inden-1-yl)carbamate is obtained in a crystalline form and has high purity, high stability, high water-solubility, and low hygroscopicity.

### TECHNICAL FIELD

The present invention relates to a novel salt of a dimethyl-2,3-dihydro-1H-indene derivative and processes for preparing the same. More specifically, the present invention relates to a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate, having improved stability, solubility, and hygroscopicity; and processes for preparing the same.

### BACKGROUND ART

Dimethyl-2,3-dihydro-1H-indene derivatives, including the compound of Formula 1 below, or a pharmaceutically acceptable salt thereof are known as a compound having excellent inhibitory activity against glucosylceramide synthase (GCS) (WO 2021/096238).

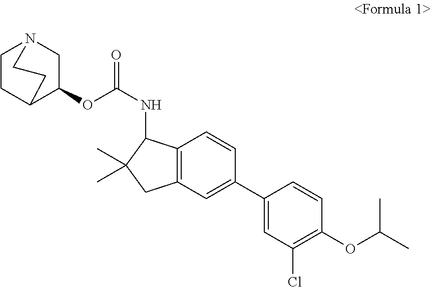 [SMILES: CC(C)Oc1ccc(-c2ccc3c(c2)CC(C)(C)C3NC(=O)O[C@@H]2CN3CCC2CC3)cc1Cl]

WO 2021/096238 discloses processes for preparing the dimethyl-2,3-dihydro-1H-indene derivatives, for example, the process as shown in Reaction Scheme 1 below.

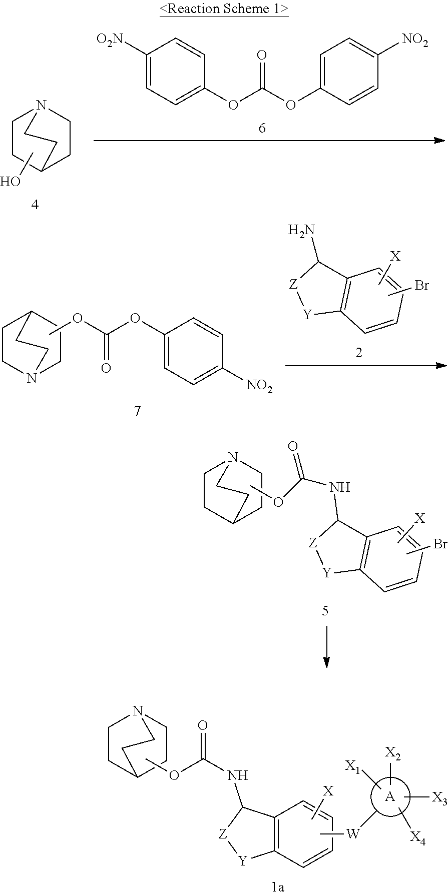

The process according to Reaction Scheme 1 comprises preparing a product (e.g., the compound of Formula 1 in the form of free base) through a Suzuki coupling reaction between the compound of Formula 5 (e.g., (S)-quinuclidin-3-yl (5-bromo-2,2-dimethyl-2,3-dihydro-1H-inden-1-yl)carbamate) and a A-W-boronic acid substituted with X₁, X₂, X₃, or X₄ (e.g., 3-chloro-4-isopropoxyphenylboronic acid) in the final step. The Suzuki coupling reaction involves the use of a palladium catalyst such as [1,1′-bis(diphenylphosphino)ferrocene]dichloropalladium(II) (Pd(dppf)Cl₂).

### DISCLOSURE OF INVENTION

### Technical Problem

The present inventors have found that the compound of Formula 1 in the form of free base form, obtained by the process disclosed in WO 2021/096238, is obtained in an amorphous form. In addition, the present inventors have also found that it is difficult to perform formulations of the compound of Formula 1 in an amorphous form, since it is obtained in low purity, exhibits low water-solubility, exhibits low stability due to showing form changes in the accelerated condition, and has high hygroscopicity.

The present inventors have carried out various studies to solve said problems. In particular, the present inventors have carried out physicochemical evaluations on various acid addition salts, in order to improve the properties (low stability, low water-solubility, and high hygroscopicity) of the compound of Formula 1 in the form of free base obtained in an amorphous form, especially a stereoisomer of the compound of Formula 1 in the form of free base obtained in an amorphous form. As a result, the present inventors have found that when the stereoisomer of the compound of formula 1 (hereinafter, also referred to as ‘(R)-compound of Formula 1’) is converted to a specific acid addition salt form (i.e., to (1S)-(+)-10-camphorsulfonic acid salt form), the salt is obtained in a crystalline form having excellent stability, high water-solubility, and low hygroscopicity in high purity.

Therefore, the present invention provides the (1S)-(+)-10-camphorsulfonic acid salt of the (R)-compound of Formula 1.

In addition, the present invention provides processes for preparing the (1S)-(+)-10-camphorsulfonic acid salt of the (R)-compound of Formula 1.

### Solution to Problem

According to an aspect of the present invention, there is provided a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate.

According to another aspect of the present invention, there is provided a process for preparing a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate, the process of which comprises: (a) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent under heating to obtain a solution; and (b) cooling the solution obtained in the step (a) to form a precipitate.

According to still another aspect of the present invention, there is provided a process for preparing a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate, the process of which comprises: (i) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent at 5° C. to 30° C. to obtain a solution; and (ii) adding an anti-solvent to the solution obtained in the step (i) to form a precipitate.

According to still another aspect of the present invention, there is provided a process for preparing a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate, the process of which comprises: (p) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent at 5° C. to 30° C. to obtain a solution; and (q) evaporating the solvent from the solution obtained in the step (p).

### Advantageous Effects of Invention

The (1S)-(+)-10-camphorsulfonic acid salt of the (R)-compound of Formula 1 obtained according to the present invention is obtained in a crystalline form having high purity. In addition, the (1S)-(+)-10-camphorsulfonic acid salt of the (R)-compound of Formula 1 has excellent stability, high water-solubility, and low hygroscopicity. Therefore, the (1S)-(+)-10-camphorsulfonic acid salt of the (R)-compound of Formula 1 according to the present invention enables effective for-mulation.

### BRIEF DESCRIPTION OF DRAWINGS

FIG. 1 shows the XRD spectrum of the (S)-quinuclidin-3-yl (5-(3-chloro-4-isopropoxyphenyl)-2,2-dimethyl-2,3-dihydro-1H-inden-1-yl)carbamate (in the form of free base) prepared according to the process disclosed in WO 2021/096238.

FIG. 2 shows the XRD spectrum of the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate obtained in the present invention (Example 1).

FIG. 3 shows the XRD spectra of the compounds obtained in Comparative Examples 1 to 3 (in the form of hydrochloride, tartrate, and citrate salts).

FIG. 4 shows the DSC thermogram of the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate obtained in the present invention (Example 1).

FIG. 5 shows the XRD spectra measured after storing the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate obtained in the present invention (Example 1) under the accelerated condition.

FIG. 6 shows the appearances observed after storing the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate obtained in the present invention (Example 1) under the accelerated condition.

### BEST MODE FOR CARRYING OUT THE INVENTION

The present invention provides a novel salt of the (R)-compound of Formula 1 having high purity, high stability, high water-solubility, and low hygroscopicity. That is, the present invention provides a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate, having the following chemical structure of Formula 1a.

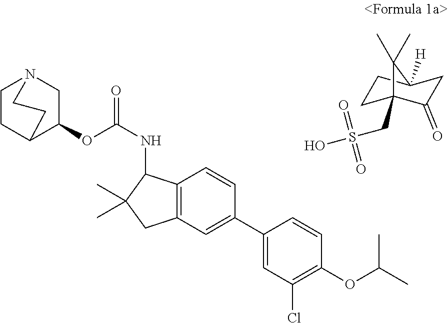 [SMILES: CC(C)Oc1ccc(-c2ccc3c(c2)CC(C)(C)C3NC(=O)O[C@@H]2CN3CCC2CC3)cc1Cl ; CC1(C)[C@@H]2CC[C@@]1(CS(=O)(=O)O)C(=O)C2]

It has been found by the present invention that the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate is obtained in a crystalline form. Therefore, the present invention also provides a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate in a crystalline form.

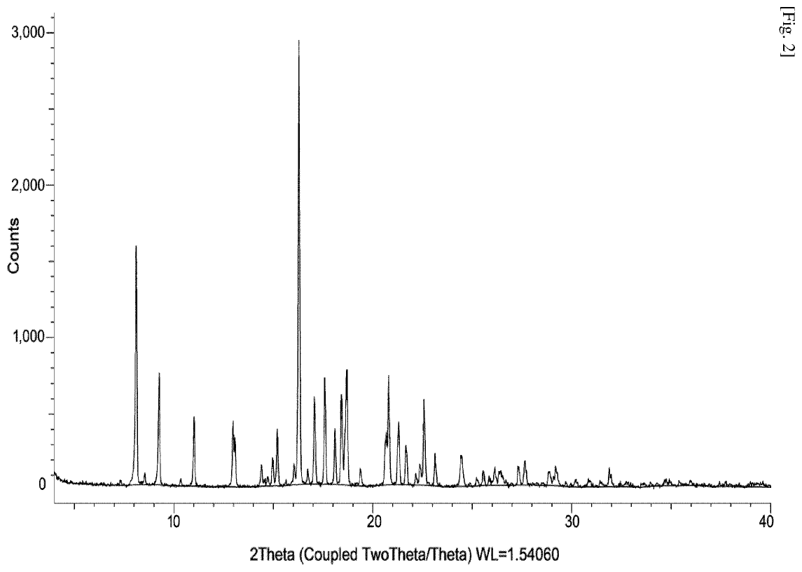

In one embodiment, the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate in a crystalline form may have an XRD pattern with peaks at 8.1, 11.0, and 16.3°2θ±0.2°2θ, preferably at 8.1, 9.3, 11.0, 16.3, and 17.6°2θ±0.2°2θ, more preferably at 8.1, 9.3, 11.0, 13.0, 13.1, 15.2, 16.3, 17.1, 17.6, 18.4, 18.7, 20.8, and 22.6°2θ±0.2°2θ. For example, the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate in a crystalline form may have the XRD pattern of FIG. 2.

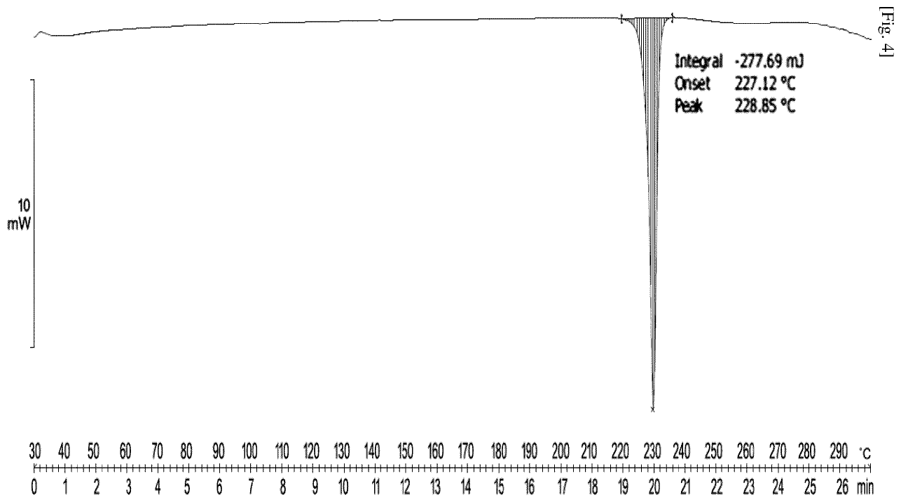

In another embodiment, the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate in a crystalline form may have a differential scanning calorimetry (DSC) thermogram showing an endothermic peak at 220-236° C., preferably at 229±2° C., for example the differential scanning calorimetry (DSC) thermogram of FIG. 4.

The present invention includes, within its scope, processes for preparing the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate.

In one embodiment, the process may comprise (a) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent under heating to obtain a solution; and (b) cooling the solution obtained in the step (a) to form a precipitate.

In said embodiment, the step (a) may be carried out at a temperature of 35° C. to 70° C., preferably 40° C. to 60° C. The solvent used in the step (a) may be a single solvent or a mixed solvent. For example, the single solvent may be selected from the group consisting of acetone, methyl ethyl ketone, C₁˜C₅ alcohol, 2-methyltetrahydrofuran, isopropyl acetate, methyl t-butyl ether, and ethyl acetate. And, for example, the mixed solvent may be a mixed solvent of water and an organic solvent selected from the group consisting of acetone, ethyl acetate, isopropyl acetate, and methyl ethyl ketone. A volume ratio of the organic solvent and water in the mixed solvent may be 3:0.1 to 30:0.1.

In said embodiment, the cooling in the step (b) may be carried out at a temperature of 0° C. to 30° C., preferably 5° C. to 25° C. The precipitate formed from the step (b), namely (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carba mate, may be isolated according to conventional methods. For example, the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate may be isolated by filtering (e.g., filtering under reduced pressure) and then drying (e.g., drying in vacuo) according to conventional methods. Therefore, in said process, the step (b) may further comprise filtering and drying the formed precipitate. If necessary, a step of washing the product in a wet cake form with the used solvent may be additionally carried out after the filtration and before the drying.

In another embodiment, the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate may be prepared by using a solvent/antisolvent at temperatures including room temperature (e.g., about 5° C.˜30° C.), without carrying out a heating step. For example, the process of the present invention may comprise (i) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent at 5° C. to 30° C. to obtain a solution; and (ii) adding an anti-solvent to the solution obtained in the step (i) to form a precipitate. The solvent used in the step (i) may be one or more selected from the group consisting of methanol, ethanol, isopropyl alcohol, and acetone. The antisolvent used in the step (ii) may be selected from the group consisting of diisopropyl ether, methyl t-butyl ether, diethyl ether, and heptane.

In another embodiment, the (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate may be prepared by using a dissolving/evaporating-crystallization method at temperatures including room temperature (e.g., about 5° C.˜30° C.), without carrying out a heating step. For example, the process of the present invention may comprise (p) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent at 5° C. to 30° C. to obtain a solution; and (q) evaporating the solvent from the solution obtained in the step (p). The solvent used in the step (p) may be one or more selected from the group consisting of methanol, ethanol, isopropyl alcohol, and acetone. The evaporating of the step (q) may be carried out according to a conventional solvent evaporation method, for example, at a temperature of 5° C. to 30° C., preferably about 20° C. to 25° C.

Hereinafter, the present invention will be described in more detail through Examples and Test Examples. However, these Examples and Test Examples are provided for illustration purposes only, and are not intended to limit the scope of the invention.

### Example 1: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (1 g, 2.07 mmol) was dissolved in ethyl acetate (20 mL), a solution of (1S)-(+)-10-camphorsulfonic acid (0.48 g, 2.07 mmol) in purified water (0.2 mL) was added thereto. The reaction mixture was heated to about 50° C., stirred for about 1 hour, cooled to about 25° C., and then stirred for about 1 hour. After the reaction mixture was filtered under reduced pressure, the resulting wet cake was washed with ethyl acetate (3 mL). The resulting solid was dried in vacuo to obtain 1.2 g of the titled compound. (Yield: 81%)

¹H-NMR (400 MHz, DMSO-d₆) δ 9.47 (s, 1H), 7.68-7.67 (d, 1H), 7.64-7.62 (d, 1H), 7.57-7.54 (m, 1H), 7.46-7.44 (m, 2H), 7.27-7.22 (m, 2H), 4.93-4.90 (m, 1H), 4.82-4.66 (m, 2H), 3.73-3.68 (m, 1H), 3.28-3.19 (m, 5H), 2.89-2.85 (d, 1H), 2.80-2.64 (m, 3H), 2.39-2.36 (d, 1H), 2.28-2.20 (m, 2H), 2.08-2.03 (m, 1H), 1.95-1.73 (m, 6H), 1.33-1.31 (d, 6H), 1.30-1.26 (m, 2H), 1.16 (s, 3H), 1.05 (s, 3H), 0.90 (s, 3H), 0.74 (s, 3H)

### Example 2: Preparation of the

((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (500 mg, 1.04 mmol) was dissolved in ethyl acetate (10 mL), a solution of (1S)-(+)-10-camphorsulfonic acid (0.24 g, 1.04 mmol) in purified water (0.04 mL) was added thereto. The reaction mixture was heated to about 45° C., stirred for about 1 hour, cooled to about 25° C., and then stirred for about 1 hour. After the reaction mixture was filtered under reduced pressure, the resulting wet cake was washed with ethyl acetate (0.6 mL). The resulting solid was dried in vacuo to obtain 470 mg of the titled compound. (Yield: 63%)

### Example 3: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (200 mg, 0.41 mmol) was dissolved in acetone (2 mL), (1S)-(+)-10-camphorsulfonic acid (100 mg, 0.41 mmol) was added thereto. The reaction mixture was heated to about 45° C., stirred for about 1 hour, cooled to about 25° C., and then stirred for about 1 hour. After the reaction mixture was filtered under reduced pressure, the resulting wet cake was washed with acetone (0.6 mL). The resulting solid was dried in vacuo to obtain 280 mg of the titled compound. (Yield: 95%)

### Example 4: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (200 mg, 0.41 mmol) was dissolved in 2-methyltetrahydrofuran (2 mL), (1S)-(+)-10-camphorsulfonic acid (100 mg, 0.41 mmol) was added thereto. The reaction mixture was heated to about 45° C., stirred for about 1 hour, cooled to about 25° C., and then stirred for about 1 hour. After the reaction mixture was filtered under reduced pressure, the resulting wet cake was washed with 2-methyltetrahydrofuran (0.6 mL). The resulting solid was dried in vacuo to obtain 206 mg of the titled compound. (Yield: 70%)

### Example 5: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (2.9 g, 0.006 mol) was dissolved in ethyl acetate (58 mL), a solution of (1S)-(+)-10-camphorsulfonic acid (1.4 g, 0.006 mol) in purified water (0.6 mL) was added thereto. The reaction mixture was heated to about 50° C., stirred for about 1 hour, cooled to about 25° C., and then stirred for about 1 hour. After the reaction mixture was filtered under reduced pressure, the resulting wet cake was washed with ethyl acetate (30 mL). The resulting solid was dried in vacuo to obtain 3.4 g of the titled compound. (Yield: 79%)

### Example 6: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (200 mg, 0.41 mmol) was dissolved in n-butanol (2 mL), (1S)-(+)-10-camphorsulfonic acid (96 mg, 0.41 mmol) was added thereto. The reaction mixture was heated to about 45° C., stirred for about 1 hour, cooled to about 25° C., and then stirred for about 1 hour. After the reaction mixture was filtered under reduced pressure, the resulting wet cake was washed with n-butanol (0.6 mL). The resulting solid was dried in vacuo to obtain 178 mg of the titled compound. (Yield: 60%)

### Example 7: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate (500 mg, 1.04 mmol) was dissolved in methyl ethyl ketone (7.5 mL) and then the resulting solution was heated to about 55° C. A solution of (1S)-(+)-10-camphorsulfonic acid (240 mg, 1.04 mmol) in methyl ethyl ketone (2.5 mL) was added to the above solution. The reaction mixture was cooled to about 25° C. and then stirred for about 1 hour. After the reaction mixture was filtered under reduced pressure, the resulting wet cake was washed with methyl ethyl ketone (1.5 mL). The resulting solid was dried in vacuo to obtain 510 mg of the titled compound. (Yield: 69%)

### Example 8: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After (S)-quinuclidin-3-yl ((R)-5-(3-chloro-4-isopropoxyphenyl)-2,2-dimethyl-2,3-dihydro-1H-inden-1-yl)carbamate (500 mg, 1.04 mmol) was dissolved in 2-propanol (10 mL), (1S)-(+)-10-camphorsulfonic acid (240 mg, 1.04 mmol) was added thereto. The reaction mixture was heated to about 55° C., stirred for about 2 hours, cooled to about 25° C., and then stirred for about 1 hour. After the reaction mixture was filtered under reduced pressure, the resulting wet cake was washed with 2-propanol (1.5 mL). The resulting solid was dried in vacuo to obtain 0.51 g of the titled compound. (Yield: 69%)

### Example 9: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

### (1) Concentrated Residue of (S)-quinuclidin-3-yl ((R)-5-(3-chloro-4-isopropoxyphenyl)-2,2-dimethyl-2,3-dihydro-1H-inden-1-yl)carbamate

A mixture of (S)-3-quinuclidinol (4.6 g, 0.036 mol), tetrahydrofuran (120 mL), and bis(4-nitrophenyl) carbonate (11.5 g, 0.038 mol) was stirred at 30° C. for 8 hours. To the reaction mixture, were added (R)-5-(3-chloro-4-isopropoxyphenyl)-2,2-dimethyl-2,3-dihydro-1H-inden-1-amine (10.0 g, 0.03 mol) and tetrahydrofuran (30 mL). The reaction mixture was refluxed under stirring for about 15 hours and then cooled to room temperature. Purified water (80 mL) was added to the reaction mixture. The reaction mixture was concentrated under reduced pressure and then 50% sodium hydroxide (6.1 g, 0.076 mol) was added thereto. The reaction mixture was stirred at room temperature for about 4 hours and then concentrated under reduced pressure. To the resulting residue, were added purified water (120 mL) and ethyl acetate (170 mL). The separated organic layer was washed twice with 5% sodium carbonate (100 mL) and then concentrated under reduced pressure to obtain 13.5 g of the concentrated residue of (S)-quinuclidin-3-yl ((R)-5-(3-chloro-4-isopropoxyphenyl)-2,2-dimethyl-2,3-dihydro-1H-inden-1-yl)carba mate. (Yield: 92%)

### (2) ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

After the concentrated residue (13.5 g, 27.9 mmol) obtained in (1) was dissolved in ethyl acetate (20 mL), (1S)-(+)-10-camphorsulfonic acid (6.5 g, 27.9 mmol) was added thereto. The reaction mixture was stirred at about 50° C. for about 1 hour, cooled to about 25° C., and then stirred for about 1 hour. After the reaction mixture was filtered under reduced pressure, the resulting wet cake was washed with ethyl acetate (30 mL). The resulting solid was dried in vacuo to obtain 18.3 g of the titled compound. (Yield: 92%)

### Example 10: Preparation of the

### ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate (1.0 g, 2.07 mmol) was dissolved in ethyl acetate (20 mL) and then the resulting solution was heated to 50° C. A solution of (1S)-(+)-10-camphorsulfonic acid (0.48 g, 2.07 mmol) in purified water (0.2 ml) was added to the above solution. The reaction mixture was stirred at about 45° C. for about 1 hour, cooled to about 25° C. and then stirred for about 1 hour. After the reaction mixture was filtered under reduced pressure, the resulting wet cake was washed with ethyl acetate (3 mL). The resulting solid was dried in vacuo to obtain 1.19 g of the titled compound. (Yield: 80%)

### Example 11: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate (2.9 g, 0.006 mol) was dissolved in isopropyl acetate (20 mL) and then the resulting solution was heated to 50° C. (1S)-(+)-10-camphorsulfonic acid (1.4 g, 0.006 mol) was added to the above solution. The reaction mixture was stirred at about 50° C. for about 1 hour, cooled to about 25° C. and then stirred for about 15 hours. After the reaction mixture was filtered under reduced pressure, the resulting wet cake was washed with ethyl acetate (30 mL). The resulting solid was dried in vacuo to obtain 3.5 g of the titled compound. (Yield: 82%)

### Example 12: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate (1.0 g, 2.07 mmol) was dissolved in isopropyl acetate (20 mL) and then the resulting solution was heated to 55° C. A solution of (1S)-(+)-10-camphorsulfonic acid (0.48 g, 2.07 mmol) in purified water (0.2 ml) was added to the above solution. The reaction mixture was stirred at about 45° C. for about 1 hour, cooled to about 25° C. and then stirred for about 1 hours. After the reaction mixture was filtered under reduced pressure, the resulting wet cake was washed with isopropyl acetate (3 mL). The resulting solid was dried in vacuo to obtain 1.29 g of the titled compound. (Yield: 87%)

### Example 13: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate (1.0 g, 2.07 mmol) was dissolved in methyl ethyl ketone (20 mL) and then the resulting solution was heated to 55° C. A solution of (1S)-(+)-10-camphorsulfonic acid (0.48 g, 2.07 mmol) in purified water (0.2 ml) was added to the above solution. The reaction mixture was stirred at about 55° C. for about 1 hour, cooled to about 5° C. and then stirred for about 1 hours. After the reaction mixture was filtered under reduced pressure, the resulting wet cake was washed with methyl ethyl ketone (3 mL). The resulting solid was dried in vacuo to obtain 1.19 g of the titled compound. (Yield: 80%)

### Example 14: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in methanol (483 mg, 10 mL) at the concentration of about 0.1M. A solution obtained by dissolving (1S)-(+)-10-camphorsulfonic acid in methanol (232 mg, 1 mL) at the concentration of about 1 M was added to the above solution. After the reaction mixture was stirred at room temperature for 1 hour, the solvent was evaporated at about 20° C. for 24 hours to obtain about 715 mg of the titled compound. (Yield: 100%)

### Example 15: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in ethanol (483 mg, 10 mL) at the concentration of about 0.1M. A solution obtained by dissolving (1S)-(+)-10-camphorsulfonic acid in methanol (348 mg, 1.5 mL) at the concentration of about 1 M was added to the above solution. After the reaction mixture was stirred at room temperature for 1 hour, diisopropyl ether (20 mL) was added thereto. The resulting precipitate was filtered and then dried at room temperature to obtain about 600 mg of the titled compound. (Yield: 84%)

### Example 16: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in ethanol (483 mg, 10 mL) at the concentration of about 0.1M. A solution obtained by dissolving (1S)-(+)-10-camphorsulfonic acid in methanol (232 mg, 1 mL) at the concentration of about 1 M was added to the above solution. After the reaction mixture was stirred at room temperature for 1 hour, methyl t-butyl ether (30 mL) was added thereto. The reaction mixture was stirred at room temperature for about 12 hours and then filtered under reduced pressure. The resulting material was washed with methyl t-butyl ether and then dried in vacuo at 40° C. to obtain about 610 mg of the titled compound. (Yield: 85%)

### Example 17: Preparation of the ((1S,4R)-7,7-dimethyl-2-oxobicyclo[2.2.1]heptan-1-yl)methanesulfonic acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in acetone (483 mg, 10 mL) at the concentration of about 0.1M. A solution obtained by dissolving (1S)-(+)-10-camphorsulfonic acid in methanol (232 mg, 1 mL) at the concentration of about 1 M was added to the above solution. After the reaction mixture was stirred at room temperature for 1 hour, methyl t-butyl ether (20 mL) was added thereto. The reaction mixture was stirred at about 5° C. for about 12 hours and then filtered under reduced pressure while maintaining the temperature at about 5° C. The resulting material was washed with methyl t-butyl ether and then dried in vacuo at room temperature to obtain about 600 mg of the titled compound. (Yield: 84%)

### Comparative Example 1: Preparation of the HCl salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in methanol (483 mg, 10 mL) at the concentration of about 0.1M. A 1M hydrochloric acid solution (36 mg, 1 mL) was added to the above solution. After the reaction mixture was stirred at room temperature for 1 hour, methyl t-butyl ether (40 mL) was added thereto. The reaction mixture was stirred for about 5 hours and then filtered under reduced pressure. The resulting material (solid) was dried in vacuo at room temperature to obtain 450 mg of the titled compound. (Yield: 87%)

### Comparative Example 2: Preparation of the tartaric acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in methanol (483 mg, 10 mL) at the concentration of about 0.1M. A 1M tartaric acid solution (150 mg, 1 mL) was added to the above solution. After the reaction mixture was stirred at room temperature for 1 hour, methyl t-butyl ether (40 mL) was added thereto. The reaction mixture was stirred for about 5 hours and then filtered under reduced pressure. The resulting material (solid) was dried in vacuo at room temperature to obtain 505 mg of the titled compound. (Yield: 80%)

### Comparative Example 3: Preparation of the citric acid salt of (S)-quinuclidin-3-yl ((R)-5-(3-chloro-4-isopropoxyphenyl)-2,2-dimethyl-2,3-dihydro-1H-inden-1-yl)carbamate

(S)-quinuclidin-3-yl ((R)-5-(3-chloro-4-isopropoxyphenyl)-2,2-dimethyl-2,3-dihydro-1H-inden-1-yl)carbamate was dissolved in methanol (483 mg, 1 mL) at the concentration of about 1M. A 1M citric acid solution (192 mg, 1 mL) was added to the above solution. After the reaction mixture was stirred at room temperature for 1 hour, methyl t-butyl ether (20 mL) was added thereto. The reaction mixture was stirred for about 5 hours and then filtered under reduced pressure. The resulting material (solid) was dried in vacuo at room temperature to obtain 530 mg of the titled compound. (Yield: 79%)

### Test Example 1: Characterizations

### (1) XRD Analysis

X-ray diffraction (XRD) analyses were carried out with a Bruker's D8 Advance X-ray powder diffractometer. The measurements at the angles ranging from 4 to 40° 20 values were performed at a scanning rate of 0.1 second per 1 step, using CuK_{α1} radiation (λ_{α1}=1.54060 Å) produced at the conditions of 40 mA and 40 kV.

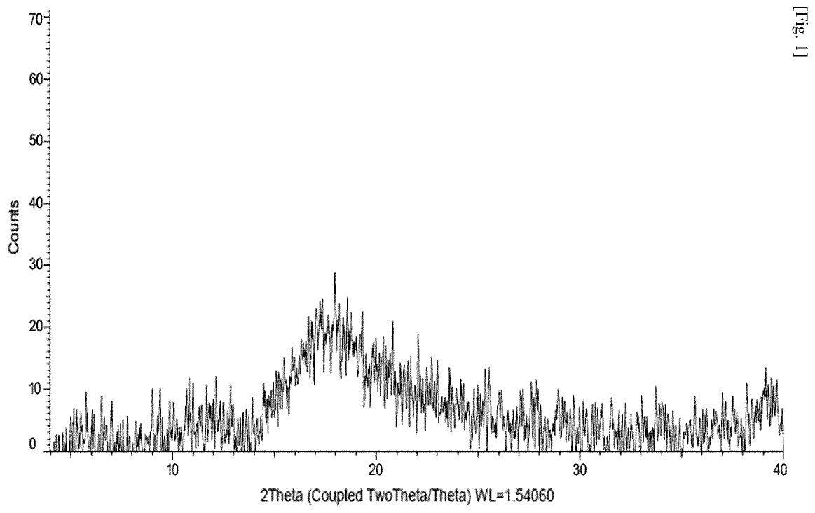

The XRD analysis result for the (S)-quinuclidin-3-yl (5-(3-chloro-4-isopropoxyphenyl)-2,2-dimethyl-2,3-dihydro-1H-inden-1-yl)carbamate (‘the compound of WO 2021/096238’, in the form of free base) prepared according to the method disclosed in WO 2021/096238 is shown in FIG. 1. From the result of FIG. 1, it can be seen that the free base prepared according to the method disclosed in WO 2021/096238 is in an amorphous form.

The XRD analysis result for the compound obtained in Example 1 is shown in FIG. 2. From the result of FIG. 2, it can be seen that the compound obtained in Example 1 is in a crystalline form. The main XRD peaks (20) of the compound obtained in Example 1 are observed at 8.1, 9.3, 11.0, 13.0, 13.1, 15.2, 16.3, 17.1, 17.6, 18.4, 18.7, 20.8, and 22.6020.

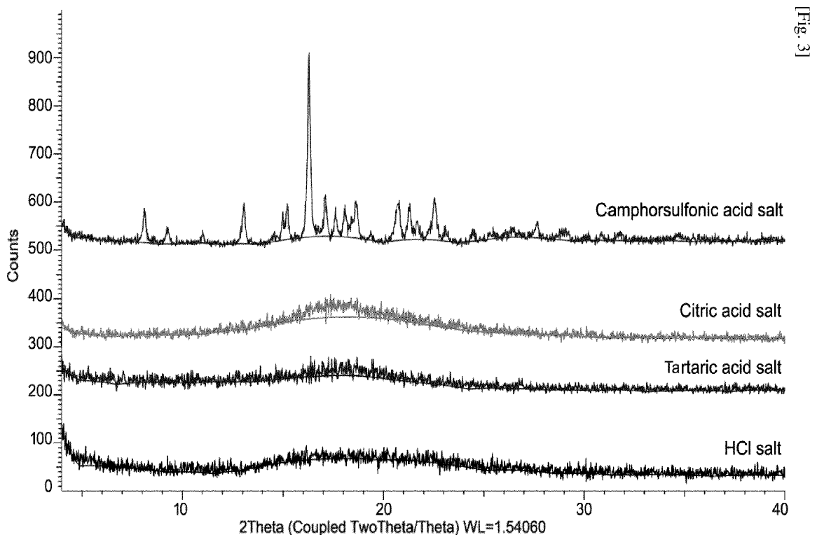

And, the XRD analysis results for the compounds obtained in Comparative Examples 1 to 3 are shown in FIG. 3. From the result of FIG. 3, it can be seen that the compounds in the form of HCl salt (hydrochloride), tartaric acid salt (tartrate) and citric acid salt (citrate) were obtained in an amorphous form.

### (2) Differential Scanning Calorimetry (DSC) Thermogram Analysis

The differential scanning calorimetry (DSC) analyses were carried out with the Mettler Toledo's DSC1 differential scanning calorimeter, under the following conditions: start temperature 30° C., end temperature 300° C., heating rate 10° C./min, and nitrogen gas flow rate 50 mL/min.

The DSC analysis result for the compound obtained in Example 1 is shown in FIG. 4. From the result of FIG. 4, it can be seen that the compound obtained in Example 1 exhibits an endothermic peak at 229±2° C.

### (3) Water Content Analysis

As a result of measuring the water content of the compound obtained in Example 1 by Karl Fischer titration, it was confirmed that the compound obtained in Example 1 had a low water content in the range of about 0.3% to 0.5%.

### Test Example 2: Stability Evaluations

### (1) Stability Test (Accelerated Condition)

Stability tests were carried out while storing the compound obtained in Example 1 in a stability test chamber (CARON), under the accelerated condition (40±2° C., 75±5% RH) for 0, 1, and 3 months. The results obtained by measuring purities (the HPLC purities) and water contents (by Karl Fischer method) are shown in Table 1 below.

TABLE 1
Compound of Example 1 ((1S)-(+)-10-camphorsulfonic acid salt)

| | Initial | 1 month | 3 months |
|---|---|---|---|
| HPLC purity | 98.6% | 98.6% | 98.6% |
| Water contents | 0.48% | 0.32% | 1.28% |

From the results of Table 1, it can be seen that the compound in the form of (1S)-(+)-10-camphorsulfonic acid salt obtained according to the present invention maintained a stable state without a change in the purity thereof under the accelerated condition. And, it can be also seen that there was no significant difference (within 1.0%) in the water contents thereof under the accelerated condition.

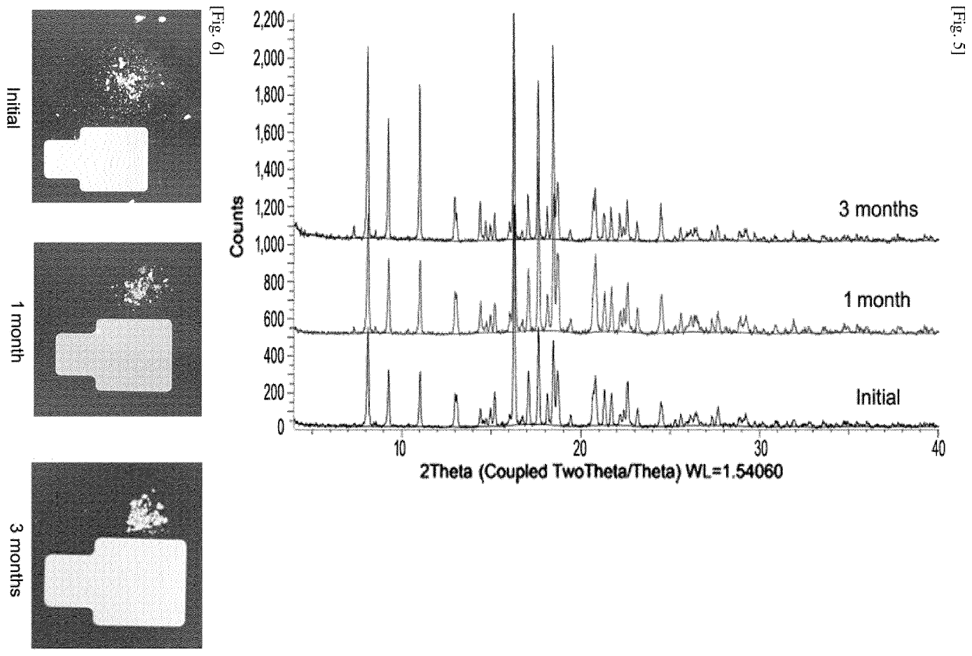

In addition, the XRD analysis results (measured by the same method as in Test Example 1) and the appearances for the compound obtained according to Example 1 under the accelerated condition as described above are shown in FIG. 5 and in FIG. 6, respectively. From the results of FIGS. 5 and 6, it can be confirmed that the compound in the form of (1S)-(+)-10-camphorsulfonic acid salt obtained according to the present invention has excellent stability under the accelerated condition.

### (2) Photostability Test

Photostability tests were carried out according to ICH guideline Q1B. That is, the photostability tests were carried out by exposing the compound obtained in Example 1 to the UV light (20 W/m²) for 10 hours and to the visible light (30,000 lux) for 40 hours, respectively. The results obtained by measuring purities (the HPLC purities) and water contents (by Karl Fischer method) are shown in Table 2 below.

TABLE 2
Compound of Example 1 ((1S)-(+)-10-camphorsulfonic acid salt)

| | | Light exposure |
|---|---|---|
| | | (UV light for 10 hours/ |
| | Initial | visible light for 40 hours) |
| HPLC purity | 98.6% | 98.6% |
| Water contents | 0.48% | 0.31% |

From the results of Table 2, it can be seen that the compound in the form of (1S)-(+)-10-camphorsulfonic acid salt obtained according to the present invention has excellent photostability.

### Test Example 3: Hygroscopicity Evaluations

Hygroscopicities for the compound obtained in Example 1 and the compounds obtained in Comparative Examples 1 to 3 were measured using a moisture adsorption analyzer (Dynamic Vapor Sorption, DVS). Specifically, the compound of WO 2021/096238, the compound obtained in Example 1, and the compounds obtained in Comparative Examples 1 to 3 were exposed in a moisture adsorption analyzer (Surface Measurement System, DVS intrinsic) under the conditions of room temperature (about 25° C.) and RH 80% for 24 hours, respectively; and then each weight change was measured. The results are shown in Table 3 below.

TABLE 3

| | Moisture adsorption |
|---|---|
| | (%) |
| Comparative Example 1 (HCl salt) | 15.6 |
| Comparative Example 2 (tartaric acid salt) | 11.6 |
| Comparative Example 3 (citric acid salt) | 12.2 |
| Example 1 ((1S)-(+)-10-camphorsulfonic acid salt) | 2.3 |

From the results of Table 3, it can be seen that the compound in the form of (1S)-(+)-10-camphorsulfonic acid salt obtained according to the present invention exhibits remarkably low hygroscopicity.

### Test Example 4: Water-Solubility Evaluations

The water-solubilities at 20° C. for the (S)-quinuclidin-3-yl (5-(3-chloro-4-isopropoxyphenyl)-2,2-dimethyl-2,3-dihydro-1H-inden-1-yl)carbamate prepared according to the method disclosed in WO 2021/096238 (‘the compound of WO 2021/096238’, in the form of free base) and the compound obtained in Example 1 were measured. The results are shown in Table 4 below.

TABLE 4

| | Water-solubility | |
|---|---|---|
| | (20° C.) | |
| Example 1 ((1S)-(+)-10-camphorsulfonic acid salt) | 1.7 | mg/mL |
| Compound of WO 2021/096238 | <0.01 | mg/mL |
| (in the form of free base) | | |

From the results of Table 4, it can be seen that the compound in the form of (1S)-(+)-10-camphorsulfonic acid salt obtained according to the present invention exhibits remarkably high water-solubility.

1. A (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate.

2. The (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate as claimed in claim 1, in a crystalline form.

3. The (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate as claimed in claim 2, wherein the crystalline form has an XRD pattern with peaks at 8.1, 11.0, and 16.3 020±0.2 020.

4. The (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate as claimed in claim 3, wherein the crystalline form has the XRD pattern of FIG. 2.

5. The (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate as claimed in claim 2, wherein the crystalline form has a differential scanning calorimetry (DSC) thermogram showing an endothermic peak at 220-236° C.

6. The (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate as claimed in claim 5, wherein the crystalline form has the differential scanning calorimetry (DSC) thermogram of FIG. 4.

7. A process for preparing a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate, the process of which comprises:
(a) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent under heating to obtain a solution; and
(b) cooling the solution obtained in the step (a) to form a precipitate.

8. The process as claimed in claim 7, wherein the step (a) is carried out at a temperature of 35° C. to 70° C.

9. The process as claimed in claim 7, wherein the step (a) is carried out at a temperature of 40° C. to 60° C.

10. The process as claimed in claim 7, wherein the solvent is an organic solvent selected from the group consisting of acetone, methyl ethyl ketone, C₁˜C₅ alcohol, 2-methyltetrahydrofuran, isopropyl acetate, methyl t-butyl ether, and ethyl acetate.

11. The process as claimed in claim 7, wherein the solvent is a mixed solvent of water and an organic solvent selected from the group consisting of acetone, ethyl acetate, isopropyl acetate, and methyl ethyl ketone.

12. The process as claimed in claim 11, wherein a volume ratio of the organic solvent and water is 3:0.1 to 30:0.1.

13. The process as claimed in claim 7, wherein the cooling is carried out at a temperature of 0° C. to 30° C.

14. The process as claimed in claim 7, wherein the cooling is carried out at a temperature of 5° C. to 25° C.

15. The process as claimed in claim 7, wherein the step (b) further comprises filtering and drying the formed precipitate.

16. A process for preparing a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate, the process of which comprises:
(i) dissolving (S)-quinuclidin-3-yl ((R)-5-(3-chloro-4-isopropoxyphenyl)-2,2-dimethyl-2,3-dihydro-1H-inden-1-yl)carbamate and (1S)-(+)-10-camphorsulfonic acid in a solvent at 5° C. to 30° C. to obtain a solution; and
(ii) adding an anti-solvent to the solution obtained in the step (i) to form a precipitate.

17. The process as claimed in claim 16, wherein the solvent is one or more selected from the group consisting of methanol, ethanol, isopropyl alcohol, and acetone.

18. The process as claimed in claim 16, wherein the anti-solvent is selected from the group consisting of diisopropyl ether, methyl t-butyl ether, diethyl ether, and heptane.

19. A process for preparing a (1S)-(+)-10-camphorsulfonic acid salt of (S)-quinuclidin-3-yl ((R)-5-(3-chloro-4-isopropoxyphenyl)-2,2-dimethyl-2,3-dihydro-1H-inden-1-yl)carbamate, the process of which comprises:
(p) dissolving (S)-quinuclidin-3-yl ((R)-5-(3-chloro-4-isopropoxyphenyl)-2,2-dimethyl-2,3-dihydro-1H-in den-1-yl)carbamate and (1S)-(+)-10-camphorsulfonic acid in a solvent at 5° C. to 30° C. to obtain a solution; and
(q) evaporating the solvent from the solution obtained in the step (p).

20. The process as claimed in claim 19, wherein the solvent is one or more selected from the group consisting of methanol, ethanol, isopropyl alcohol, and acetone.
